(19) United States
(12) Patent Application Publication
(10) Pub. No.: US 2026/0066080 A1
(43) Pub. Date: Mar. 5, 2026

(54) INFUSION PUMP SYSTEM AND ASSOCIATED OPERATING METHOD

(71) Applicant: B. Braun Melsungen AG, Melsungen (DE)

(72) Inventors: Jan Schwarz, Melsungen (DE); Sebastian Moeller, Hessisch Lichtenau (DE); Christian Mauro, Fritzlar (DE); Christoph Erlen, Kassel (DE); Maria Buerger, Malsfeld (DE); Stefan Scheler, Germering (DE); Philipp Burghardt, Muenchen (DE)

(21) Appl. No.: 19/314,208

(22) Filed: Aug. 29, 2025

(30) Foreign Application Priority Data
Sep. 5, 2024 (EP) 24198644.7

(51) Int. Cl. G16H 20/17; A61M 5/14; A61M 5/172

(52) U.S. Cl. CPC G16H 20/17; A61M 5/1413; A61M 5/172; A61M 2205/3561; A61M 2205/3584; A61M 2209/086

(57) ABSTRACT

An infusion pump system has a plurality of infusion pumps assigned to a network. Each infusion pump has an input unit for entering therapy-relevant patient data and/or system data, and a communication interface for communication with other infusion pumps in the network. A method of operating the infusion pump system includes the steps of entering therapy-relevant patient data and/or system data on one of the infusion pumps, and automatically transmitting that therapy-relevant patient data and/or system data to all of the other infusion pumps in the network to synchronize the therapy-relevant patient data and/or system data in the network.

### CROSS-REFERENCE TO RELATED APPLICATION

This application claims priority under 35 U.S. C. § 119 to European Application No. 24198644.7, filed on Sep. 5, 2024, the content of which is incorporated by reference herein in its entirety.

### FIELD

The present disclosure relates to a method for operating an infusion pump system. It also relates to an infusion pump system suitable for carrying out the method.

### BACKGROUND

EP 2954483 A1 describes an infusion pump management system in which an operator can associate a medical device (e.g. an infusion pump) with a particular entity such as a patient, a location, other medical devices or a caregiver. This association is controlled by a management resource that collects and stores relevant medical information. Once the association is made, the medical information is accessible to the infusion system and the operator, allowing for more efficient management and use of the system.

### SUMMARY

The present disclosure has the task of creating an infusion pump system and an associated operating method that enables or supports the input and synchronization of therapy-relevant patient data and system data between several infusion pumps of a network, whereby data integrity and data consistency are to be ensured in particular. The concept should be applicable for different referral methods. This should enable a patient to switch easily from one infusion pump in the network to the next.

The above task is solved by a method, infusion pump, and infusion pump system as described in the present disclosure. The infusion pump is suitable for carrying out the method and for integration into the system. Thus, also disclosed is the use of the infusion pump in the method according to the present disclosure, as well as the use of the infusion pump in the system according to the present disclosure. For use in the system, the infusion pump can be included in the system and hence can be assigned to the network as one of the plurality of infusion pumps.

Accordingly, the present disclosure teaches a method for operating an infusion pump system having a plurality of infusion pumps which are assigned to a network, wherein the respective infusion pump has an input unit for entering therapy-relevant patient data and/or system data and a communication interface for communicating with other infusion pumps of the network, wherein therapy-relevant patient data and/or system data entered at one of the infusion pumps is automatically transmitted to all other infusion pumps of the network and thereby synchronized in the network.

Accordingly, the present disclosure relates in particular to a method for synchronizing patient data between two or more infusion pumps in order to be able to enter therapy-relevant patient data on only one infusion pump and use the same patient data set on other infusion pumps.

In this context, patient data relevant to therapy are advantageous: Age, height, weight, gender, body surface area and/or the administration of opioids. Depending on the emphasis or weighting, one can also speak of patient-relevant therapy data.

For this approach, it is useful to have knowledge of the infusion pumps assigned to a patient. This means that the assignment of the infusion pumps to a network is preferably patient-oriented or patient-based.

In a first preferred variant, the infusion pumps assigned to a patient are assigned by means of an organization system (rack) into which the infusion pumps are inserted (the infusion pump according to the present disclosure accordingly can be insertable into a rack and hence can be adapted for insertion into a rack). They thus form an infusion workstation that is used for one patient. The rack preferably provides a communication platform between the plugged-in infusion pumps. A preferred solution comprises optical/electrical communication, which is advantageously implemented by means of an infrared signal. Other options via a wired solution or a radio connection (e.g. mobile radio) are possible. A communication interface can be integrated into the rack.

In a second preferred variant, the infusion pumps can be assigned to a patient via a server application and thereby allocated to the network, i.e. the infusion pumps are assigned a unique identification feature that represents a patient, for example. For this second variant, it is necessary for the infusion pumps to have a communication unit that communicates with the server application. The communication is preferably wireless (e.g. WLAN or Bluetooth), but could also be wired.

In a third preferred variant, the infusion pumps can be connected via an ad-hoc network, i.e. an infusion pump networks with other infusion pumps via a wireless communication interface (e.g. WLAN or Bluetooth) and in this way forms a network of several infusion pumps, which together form an infusion workstation.

The three assignment variants mentioned (rack, server, ad-hoc) can also be combined with each other or realized simultaneously. For example, an ad-hoc network can be imitated if a server fails, or a server in combination with a rack defines the network, or a first ad-hoc network is connected to a second ad-hoc network or a single infusion pump via a server.

As soon as an entry is made on an infusion pump that belongs to this network, the other infusion pumps are blocked from entering therapy-relevant patient data. At the same time, the therapy-relevant patient data is forwarded to the other infusion pumps so that they have the newly entered data available.

When transferring or transmitting the data, it is useful to distinguish between two cases: a.) an infusion pump is in use with the patient, i.e. it is infusing (“delivering”), b.) an infusion pump is not in use. If case a.) occurs, the therapy-relevant patient data is temporarily stored in the infusion pump (receiving the input) and, if necessary, updated based on further inputs. They are only applied once the current therapy has been completed or ended. In other words, this means that the temporarily stored data is updated if a parameter is updated a second (or second/third/fourth/ . . . ) time on another infusion pump during temporary storage. In case b.), the therapy-relevant patient data is preferably transferred directly and is available to the user or the (other) infusion pump(s).

In other words, in case a) the newly entered data is applied with a delay after completion of the current infusion and in case b) without any significant delay and in this sense “immediately”.

In both cases, however, a verification or plausibility check can be provided before the data is used, in which the received data is checked for validity by means of a checksum. In addition, time stamps and/or sequence numbers are exchanged in order to check the plausibility of the received data. If, for example, the received time stamps or sequence numbers are “out of date”, the received data is discarded.

It is generally possible to interrupt the input of therapy-relevant patient data or patient-relevant patient data or to continue to another infusion pump in the network. The latter is necessary if a form of therapy requires further patient-relevant therapy data that was not previously entered/available. In this case too, it is preferable that the simultaneous input of patient-relevant therapy data to other infusion pumps in the network is blocked, whereby the patient-relevant therapy data is then sent or transferred to the other infusion pumps in the network (alternatively directly, if there is no blocking).

Furthermore, patient-relevant therapy data can be deleted in the entire network. This event can be triggered on an individual infusion pump, but it is advisable to trigger it in stages, as in cases a.) and b.) above. This means that an infusion pump that has an active or running application resets its patient-relevant therapy data only after an application has ended, while an infusion pump that is not in an application resets its patient-relevant therapy data immediately.

In general, this concept can not only be limited to the exchange of patient-relevant therapy data, but it is also possible to exchange and synchronize generally valid data in a network. Such data includes, for example, the assignment to a bed location, which is confirmed once by a user at a pump in the network (bedplace assignment/location assignment) or the selection of a care unit in a hospital or similar medical facility, which is also distributed and synchronized throughout the entire network. It is also possible to make individual settings/configuration data, such as pressure level settings, language selection information, brightness settings, volume settings, time and date settings, the activation/deactivation of a WLAN module or the activation/deactivation of barcode information or Keep-Vein-Open (KVO) therapy on an infusion pump and distribute them in the network.

The input unit does not necessarily have to be part of the respective infusion pump. Input via a central input unit, a coupled device (such as a smartphone or tablet) or another infusion pump in the network is also conceivable. The input unit within the meaning of the present disclosure is therefore generally an input unit assigned or assignable to the infusion pump. In particular, the input unit can also be a decentralized input unit that is not a physical component of the infusion pump. For example, a centralized or transportable input unit.

In summary, the present disclosure relates to a method for synchronizing patient data and/or system data between two or more infusion pumps. If one or more therapy-relevant patient data and/or system data are entered at an infusion pump, this information is forwarded by the infusion pump at which the entry is made to other infusion pumps assigned to the patient. Assignment to a patient can take place by means of an arrangement in an organization system (rack) or by grouping infusion pumps into a common network via a server application or an ad hoc network.

What has been said with regard to the method applies analogously to the infusion pump system and vice versa. Accordingly, aspects and features of the method are applicable analogously to the infusion pump system. Thus, the infusion pump system according to the present disclosure having a plurality of infusion pumps which are assigned to a network, the respective infusion pump having an input unit for entering therapy-relevant patient and/or system data and a communication interface for communication with other infusion pumps in the network, the respective infusion pump having a controller which automatically transmits therapy-relevant patient data and/or system data entered at the input unit to all the other infusion pumps in the network and thereby synchronizes them in the network, can in particular have one or more of the following characteristics:
i. infusion pumps are assigned to the network by a rack into which the infusion pumps are plugged, wherein optionally the communication interface is integrated into the rack;
ii. an assignment of infusion pumps to the network is carried out by a server, which manages the infusion pumps and preferably assigns specific patients to them;
iii. an assignment of infusion pumps to the network is performed by an ad hoc network initiated by one of the infusion pumps;
iv. the therapy-relevant patient data comprises age, height, weight, gender, body surface area of a patient and/or administration of opioids;
v. the system data comprises configuration settings such as pressure level settings, language selection information, brightness settings, volume settings, time and date settings, enabling/disabling a WLAN module, enabling/disabling barcode information, and/or enabling/disabling a keep-vein-open (KVO) therapy;
vi. the system data comprises the assignment of an infusion pump to a bed location and/or the selection of a care unit within a hospital;
vii. during the input and transmission of therapy-relevant patient data and/or system data to/from one of the infusion pumps, all other infusion pumps of the network are blocked for the input of therapy-relevant patient data and/or system data;
viii. therapy-relevant patient data and/or system data entered at one of the infusion pumps during an ongoing infusion are temporarily stored there and are only used after the end of the infusion;
ix. outside a running infusion the transmission takes place immediately after input of the therapy-relevant patient data; and/or
x. patient-relevant therapy data are deleted in the entire network after corresponding user input, and wherein an infusion pump which is in an application running resets its patient-relevant therapy data only after the application has ended, while an infusion pump which is not in any application resets its patient-relevant therapy data immediately.

### BRIEF DESCRIPTION OF THE DRAWING FIGURES

Various embodiments of the present disclosure are explained in more detail with reference to the accompanying drawings.

FIG. 1 shows a group of infusion pumps in a rack;

FIG. 2 shows a network of infusion pumps in a client-server architecture;

FIG. 3 shows a network of infusion pumps in an ad-hoc network; and

FIGS. 4-11 show various screenshots of a graphical user interface of an infusion pump associated with a network.

### DETAILED DESCRIPTION

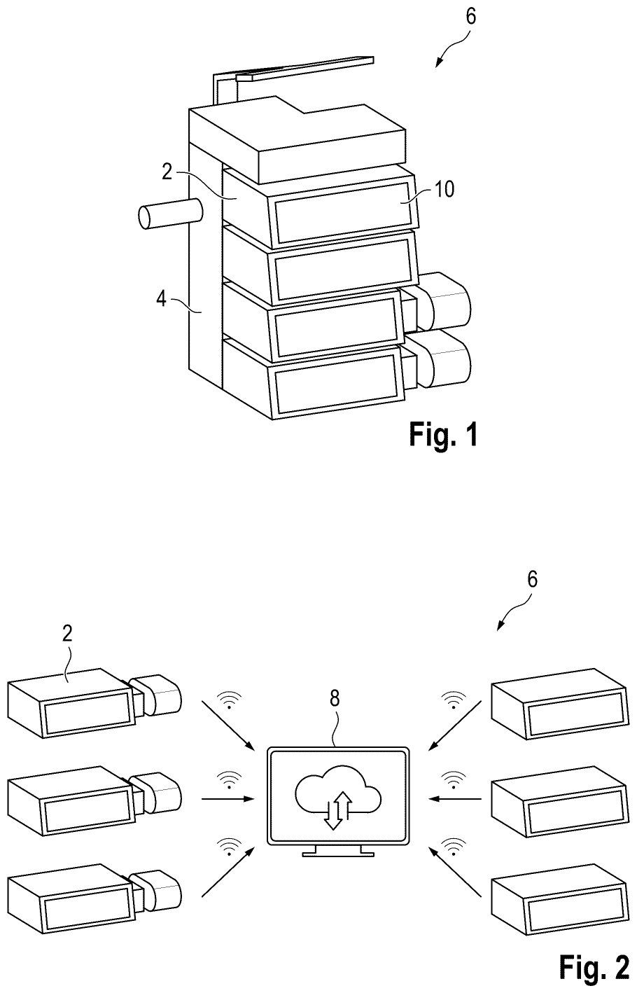

FIG. 1 shows a perspective view of a plurality of different medical infusion pumps 2, which are plugged into a common rack 4 and thus connected to form a network (infusion pump system 6). The infusion pumps 2 each have communication interfaces through which they can communicate with each other within the rack 4. Data is transmitted via data lines integrated in the rack 4, optical transmitters (infrared) and plug-in connections.

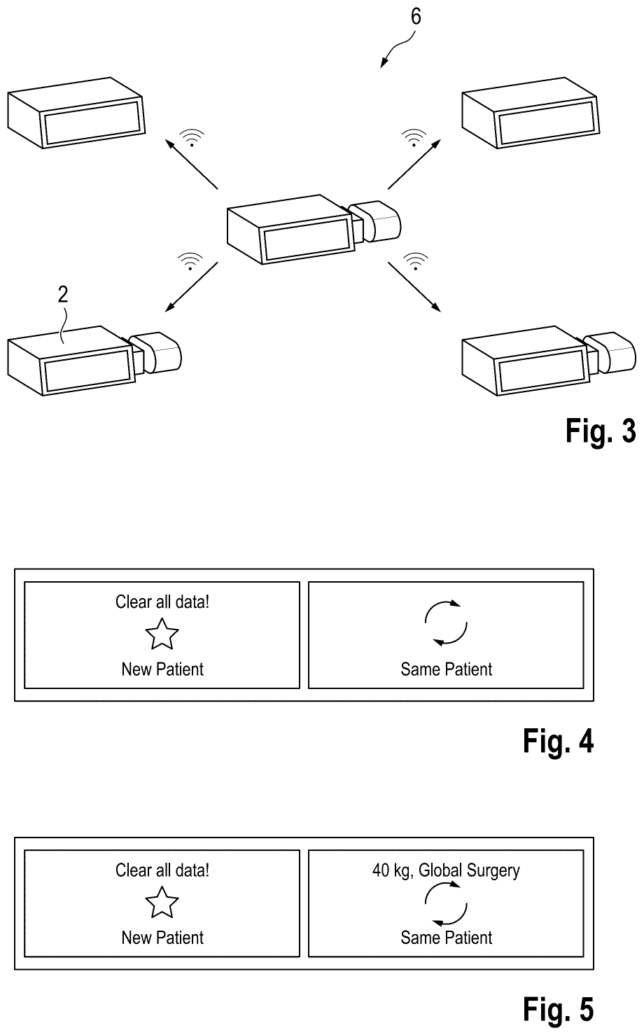

An alternative network of communicating infusion pumps 2 is shown schematically in FIG. 2. The infusion pumps 2 form end devices of a client-server network with a central server 8, which controls the assignment of the infusion pumps 2 to the network and the communication between them (the network topology does not necessarily have to be star-shaped, however).

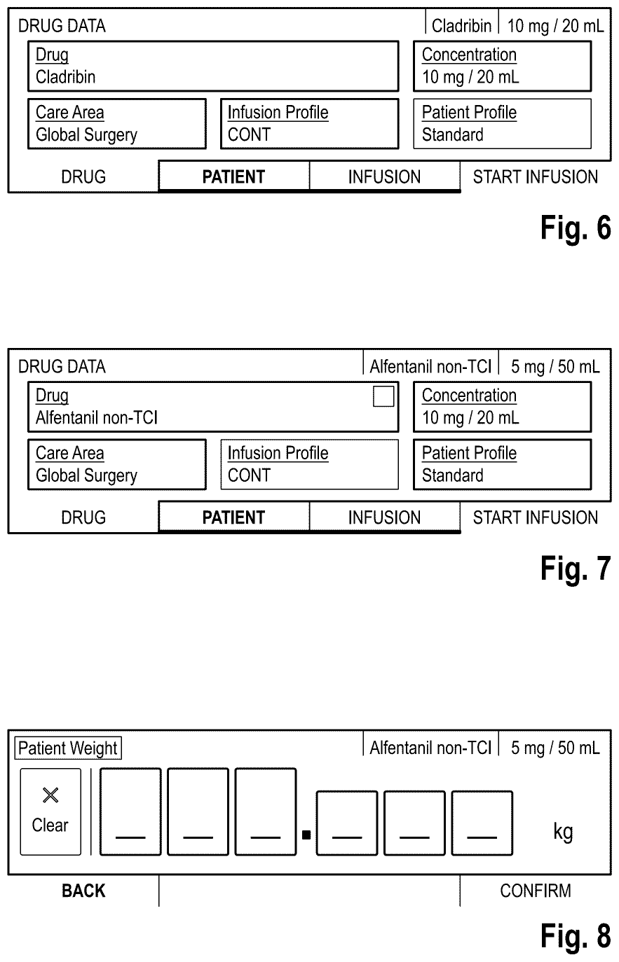

In the variant shown in FIG. 3, several infusion pumps 2 are grouped together in the form of an ad hoc network communicating with each other. An ad hoc network is a decentralized network in which the participating devices communicate with each other directly and without a central infrastructure or fixed routers (peer-to-peer communication). The devices recognize and connect to each other automatically. Thanks to a dynamic topology, the devices can in principle move freely and the network can adapt accordingly.

According to the present disclosure, in each of the three scenarios, automatic transmission or transfer or forwarding (preferably by “push” or by broadcast or alternatively by unicast) of therapy-relevant patient data and/or system data, which are entered at one of the infusion pumps 2 via an input unit 10, to the other infusion pumps 2 of the network is provided, so that a system-wide distribution and synchronization of this data takes place. The automatic synchronization of the data ensures that all infusion pumps 2 in the network always have up-to-date therapy-relevant information, which improves the efficiency and safety of the therapy.

The integrity and consistency of the synchronized data is advantageously ensured by blocking other infusion pumps 2 during the input and transmission of data, as well as by intermediate storage and later transmission during ongoing infusions.

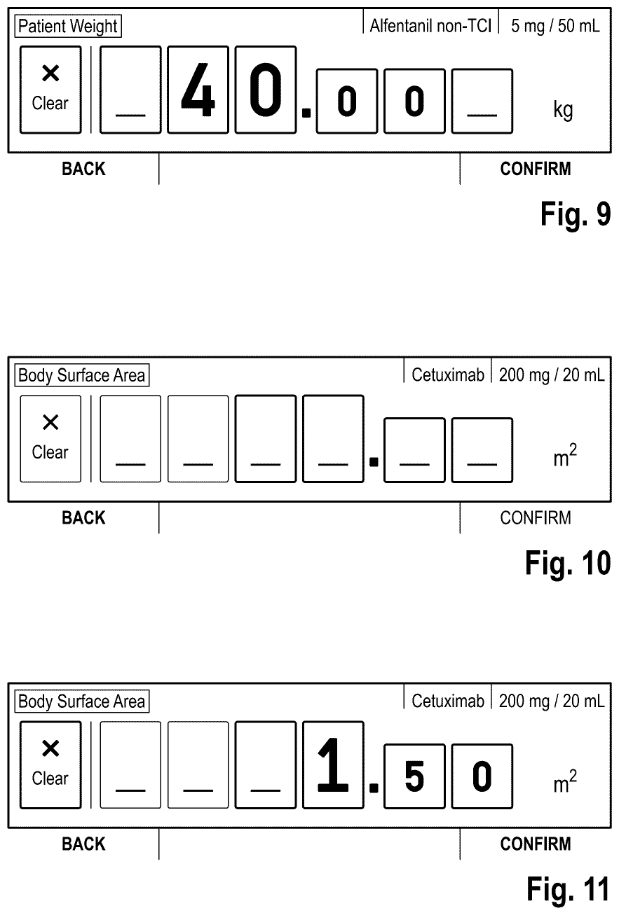

The process of data input and forwarding is illustrated purely by way of example using FIGS. 4-11, which show various screenshots of a graphical user interface (e.g. touch screen as realization of an input unit 10) of an infusion pump 2 selected for data input.

In FIG. 4, the user taps on “New patient” in the menu, which starts the data entry and at the same time blocks the data entry on the other infusion pumps 2 in the network. FIG. 5 shows the case of a patient who has already been entered with default values already stored or pre-filled in the corresponding input screens.

In the following dialog, the medication or infusion solution to be administered (here: Cladribine) is selected from a selection list according to FIG. 6, which also contains information on the concentration and the (time) infusion profile (here: continuous). FIG. 7 shows a possible alternative selection (here: Alfentanil).

Tapping on “Patient” takes the user to an input screen shown in FIGS. 8 and 9, in which the necessary, therapy-relevant patient data for the selected medication is requested (here for the medication Alfentanil: weight of the patient in kg). FIG. 8 shows the still empty input screen, FIG. 9 the input screen completed by the user.

The entered value is accepted by tapping on “Confirm”. The entered value is then transferred to the other infusion pumps 2 in the network and thus synchronized system-wide. This means that this value is used for a subsequent infusion, regardless of which infusion pump 2 in the group is ultimately used. Once synchronization is complete, the input is unlocked again on the other infusion pumps 2.

FIGS. 10 and 11 show an alternative query and input (here for the drug Cetuximab: body surface area in m²).

Of course, a patient data set to be recorded and transmitted can also include more than one query/input or any combination thereof.

If the infusion pump on which the patient data is entered is in an active infusion mode during the input, the input is temporarily stored there and, if necessary, updated on the basis of further inputs.

In a preferred implementation, this means that if a parameter set is changed on an infusion pump that is already active or “pumping”, this is always transferred to all other infusion pumps (and also adopted on the pump on which the input is made). The other infusion pumps adopt this value immediately if there is no active therapy (pumping), otherwise the update takes place after the end of therapy.

The deletion of a patient data set or one or more values from it is treated in principle like an input and carried out as described above.

### REFERENCE CHARACTER LIST

2 Infusion pump
4 Rack
6 Infusion pump system
8 Server
10 Input unit

1. A method for operating an infusion pump system with a plurality of infusion pumps assigned to a network, each infusion pump having an input unit for inputting therapy-relevant patient data and/or system data and a communication interface for communication with other infusion pumps assigned to the network, the method comprising the steps of:
inputting therapy-relevant patient data and/or system data at one of the plurality of infusion pumps; and
automatically transmitting the therapy-relevant patient data and/or system data to the other infusion pumps assigned to the network to synchronize the therapy-relevant patient data and/or system data in the network.

2. The method according to claim 1, wherein infusion pumps are assigned to the network by a rack into which the plurality of infusion pumps are plugged.

3. The method according to claim 2, wherein the communication interface is integrated into the rack.

4. The method according to claim 1, wherein an assignment of infusion pumps to the network is carried out by a server that manages the plurality of infusion pumps.

5. The method according to claim 4, wherein the server assigns specific patients to the plurality of infusion pumps.

6. The method according to claim 1, wherein an assignment of infusion pumps to the network is performed by an ad hoc network initiated by one of the plurality of infusion pumps.

7. The method according to claim 1, wherein the therapy-relevant patient data comprises age, height, weight, gender, body surface area of a patient and/or administration of opioids.

8. The method according to claim 1, wherein the system data comprises configuration settings, the configuration settings including one or more of pressure level settings, language selection information, brightness settings, volume settings, time and date settings, enabling/disabling a WLAN module, enabling/disabling barcode information, and/or enabling/disabling a keep-vein-open therapy.

9. The method according to claim 1, wherein the system data comprises an assignment of an infusion pump to a bed location and/or a selection of a care unit within a hospital.

10. The method according to claim 1, wherein, during the steps of inputting therapy-relevant patient data and/or system data at one of the plurality of infusion pumps and automatically transmitting the therapy-relevant patient data and/or system data to the other infusion pumps, inputting of therapy-relevant patient data and/or system data is blocked at the other infusion pumps.

11. The method according to claim 1, wherein therapy-relevant patient data and/or system data entered at one of the plurality of infusion pumps during an infusion are temporarily stored at said one of the plurality of infusion pumps and used only after the infusion ends.

12. The method according to claim 1, wherein, outside a running infusion, the therapy-relevant patient data and/or system data is transmitted immediately after being inputted.

13. The method according to claim 1, wherein:
patient-relevant therapy data are deleted from the network after a corresponding user input,
each infusion pump that is running an application resets patient-relevant therapy data after the application has ended, and
each infusion pump that is idle resets its patient-relevant therapy data immediately.

14. An infusion pump system comprising a plurality of infusion pumps assigned to a network, each infusion pump comprising:
an input unit for entering therapy-relevant patient and/or system data;
a communication interface for communication with other infusion pumps assigned to the network; and
a controller configured to automatically transmit therapy-relevant patient data and/or system data entered at the input unit to the other infusion pumps assigned to the network so that the therapy-relevant patient data and/or system data are synchronized in the network.

15. The infusion pump system according to claim 14, wherein each infusion pump is configured for plugging into a rack.

16. The infusion pump system according to claim 15, wherein the infusion pump system comprises the rack.

17. The infusion pump system according to claim 16, wherein the communication interface is integrated into the rack.

18. The infusion pump system according to claim 14, further comprising a server configured to assign the plurality of infusion pumps to the network.

19. The infusion pump system according to claim 18, wherein the server is configured to assign specific patients to the plurality of infusion pumps.

20. An infusion pump configured to be assigned to a network of infusion pumps, the infusion pump comprising:
an input unit for inputting therapy-relevant patient data and/or system data;
a communication interface for communication with other infusion pumps assigned to the network of infusion pumps; and
a controller configured to automatically transmit therapy-relevant patient data and/or system data input at the input unit to the other infusion pumps assigned to the network of infusion pumps and synchronize the therapy-relevant patient data and/or system data in the network of infusion pumps.
